(19) United States
(12) Patent Application Publication
(10) Pub. No.: US 2022/0111346 A1
(43) Pub. Date: Apr. 14, 2022

(54) REACTOR CASCADE AND METHOD FOR OPERATING A REACTOR CASCADE

(71) Applicant: Siemens Energy Global GmbH & Co. KG, München (DE)

(72) Inventors: Manfred Baldauf, Erlangen (DE); Frank Hannemann, Röttenbach (DE); Katharina Meltzer, Erlangen (DE); Marc Sattelberger, Nürnberg (DE); Alexander Tremel, Möhrendorf (DE)

(73) Assignee: Siemens Energy Global GmbH & Co. KG, Munich, Bayern (DE)

(21) Appl. No.: 17/424,339

(22) PCT Filed: Jan. 28, 2020
(86) PCT No.: PCT/EP2020/052002

§ 371 Date: Jul. 20, 2021

(30) Foreign Application Priority Data
Jan. 30, 2019 (DE) 10 2019 201 172.1

(51) Int. Cl. B01J 8/06; C07C 31/04; C07C 29/152; C07C 29/151

(52) U.S. Cl. CPC B01J 8/065; C07C 31/04; C07C 29/152; C07C 29/1512; B01J 2208/06; B01J 2208/00283; B01J 2208/00539; B01J 2208/00893; B01J 2208/027; B01J 2208/00176

(57) ABSTRACT

A reactor cascade for carrying out equilibrium-limited reactions, having at least two reactor units with in each case one reaction part in the form of a tubular reactor and in each case one absorption part. The reaction part has a starting product inlet and the absorption part has a starting product outlet for the discharge of excess starting products. A connecting line is provided between the starting product outlet of a first reactor unit and the starting product inlet of a second reactor unit. A pressure reduction valve for the reduction of a process pressure is provided between the first reaction unit and the second reactor unit.

### CROSS REFERENCE TO RELATED APPLICATIONS

This application is the US National Stage of International Application No. PCT/EP2020/052002 filed 28 Jan. 2020, and claims the benefit thereof. The International Application claims the benefit of German Application No. DE 10 2019 201 172.1 filed 30 Jan. 2019. All of the applications are incorporated by reference herein in their entirety.

### FIELD OF INVENTION

The invention relates to a reactor cascade and to a method of operating a reactor cascade.

### BACKGROUND OF INVENTION

Fossil energy carriers cause carbon dioxide emissions that are not in accordance with the global climate protection aims. Alternative renewable energy sources generate power, but this is not available in the same output at all times, i.e. is subject to variation. There is currently a search for approaches for viable utilization of this available renewably generated electrical power and for production of chemical products of value, for example. One means is the electrochemical conversion of water to hydrogen and oxygen. The hydrogen produced can then react with carbon dioxide as starter molecule, which would simultaneously reduce carbon dioxide emissions. Carbon dioxide, which is relatively readily available and should not be emitted into the atmosphere in any case, can thus be utilized as an inexpensive carbon source. For example, methanol is a possible product of a one-stage synthesis from carbon dioxide and hydrogen according to the following equation:

CO₂+3H₂->CH₃OH+H₂O

A disadvantage of the synthesis of methanol from carbon dioxide and hydrogen is low equilibrium conversions, which are only about 20% at 50 bar and 250 degrees Celsius. Therefore, a large portion of the gaseous reactants has to be circulated. As a result of the pressure drops that occur in a reactor, the gas has to be recompressed each time for the purpose, which is very energy-intensive and distinctly reduces the efficiency of the process. As well as these energy disadvantages, such a gas recycle process run in circulation is only of limited suitability for dynamic operation of the plant, which is particularly unfavorable especially given the fluctuating power sources of the renewable energy sources.

A continuous process regime is described in DE 102016210224 A1, in the form of a stirred tank. Based on reactor volume, however, stirred tank reactions are costlier than tubular reactors, particularly at high pressures. Furthermore, the capacity thereof is limited depending on the pressure. For that reason, they are not very suitable for industrial scale conversions of carbon dioxide and hydrogen to methanol. Moreover, stirred reactors, by comparison with tubular reactors, contain moving components that generally entail a higher level of maintenance.

### SUMMARY OF INVENTION

It is an object of the invention to provide a continuous process for synthesis of equilibrium-limited reactions that requires lower energy expenditure, i.e. higher efficiency, compared to the prior art, while entailing a lower level of maintenance compared to the prior art coupled with suitability for industrial-scale processes.

The object is achieved by a reactor cascade for implementation of equilibrium-limited reactions, and by a process for performing an equilibrium-limited reaction.

The inventive reactor cascade for implementation of equilibrium-limited reactions has at least two reactor units, each in the form of a tubular reactor. Each of the reactor units comprises a reaction section and an absorption section. The reaction section in turn has a reactant inlet, and the absorption section has a reactant outlet for leading off excess reactants. There is a connecting conduit between the reactant outlet of a first reaction unit and the reactant inlet of a second reactor unit. This connecting conduit has been provided with a pressure reduction valve for reduction of a process pressure p between the first reaction unit and the second reaction unit.

The present invention enables onward passage of excess products without further processing, especially without further energy-intensive compression, into a further reactor unit of the same type, in which the same reaction can be continued, merely with slightly altered reduced pressure conditions. The reduction in the pressure conditions only slightly affects the efficiency of the equilibrium-limited reaction that takes place in the second reaction unit. It is possible here to use a tubular reactor of inexpensive construction that does not require any moving parts and hence entails low maintenance complexity. Moreover, this reaction cascade is of especially good suitability for employment in continuous processes.

Compared to the prior art, it is also appropriate when the tubular reactor section of the reaction unit is configured such that the reactant inlet is provided at one end of the tubular reactor section and the absorption section is disposed at the other end of the reactor. The absorption section is advantageously flanged onto the reaction section at this point by a flange. This construction also contributes to a simplification of construction and hence to an inexpensive production of the reaction cascade.

The separation of the reaction unit into a reaction section and into an absorption section also has the advantage that the absorbent is present exclusively in the spatially separated absorption section, and hence contact of the absorbent with the catalyst materials present in the reaction section is avoided. Contact of the catalyst material and the absorbent would distinctly lower the efficiency of the reaction and the efficacy of the catalyst. For this purpose, a gas filter apparatus in particular is suitable. The absorption section arranged is.

The term “tubular” is understood here to mean an elongate structure which is hollow in the middle and has an aspect ratio greater than three, advantageously greater than six, more advantageously greater than eight. The cross section of the tubular reactor housing is advantageously round or oval, although other cross sections, for example rectangular or square cross sections, are also regarded as being tubular.

Moreover, the reaction cascade is advantageously configured in such a form that the absorption section of the reaction unit also has an absorbent outlet as well as the reactant outlet. The absorbent outlet is advantageously connected to a desorption unit, such that the discharged product-laden absorbent can be separated therefrom in the desorption unit, and then the processed or unladen absorbent can be introduced back into the absorption section in an inexpensive manner.

Moreover, this permits configuration of the reaction cascade described such that the respective reaction units are configured in the same way in terms of their principle of construction and in terms of their shaping. What is meant by “in the same way” is that a advantageously upright tubular reactor is provided in each case, to the lower portion of which the absorption section is attached or connected by flange. What is also meant by “in the same way” is that the individual reaction units may in principle be of reduced volume along the cascade, especially in the form of their reaction volume in the reaction section. In this case, however, they merely have a shrunken geometry; the configuration remains the same. The reason for the advantageous shrinkage of the reaction volume from the first reaction unit to the second reaction unit is that less excess reactant gas is taken out of the first reaction unit and led off than is originally introduced into the first reaction unit. It is thus possible to configure the second reaction unit and the downstream further reaction unit in a smaller and hence less expensive manner.

A further constituent of the invention is a process for performing an equilibrium-limited reaction. In this process, reactants are guided into a reaction section of a reaction unit, wherein the reaction section is at least partly filled with a porous catalytic substance. The gaseous reactants flow through this catalytic substance, with at least partial conversion of the reactant(s) to one or more reaction products at a surface of the catalytic substance. Subsequently, the reaction product and excess reactant are guided from the reaction section into an absorption section of the reaction unit, where the reaction product is absorbed by an absorbent. Excess gaseous reactant is separated from the reaction product by means of a gas filter apparatus. There is a pressure p1 in the reaction unit described. It is a feature of the invention that the separated reactant is guided through a pressure reduction apparatus and introduced into a second reaction unit at a pressure p2 lower than the pressure p1.

The advantage of the inventive process described, analogously to the advantages already described for the inventive apparatus, is that a continuous reaction of equilibrium-limited reactions can take place. At the same time, it is possible to dispense with the use of moving parts in the reaction unit, and the process described and the reaction cascade described are suitable for large inputs.

Reference is made here to a reactant which is introduced into the reaction section. In principle, the conversion of a single chemical substance over a catalyst surface to one or more reaction products is possible. In an advantageous configuration form of the invention, however, a reactant or reactant gas that comprises both carbon dioxide and hydrogen is introduced, and hence consists of at least two chemical compounds. The reaction product formed may be one or more chemical compounds; in the synthesis already described, the carbon dioxide and hydrogen reactants, given suitable choice of the catalyst, form methanol as reaction product. The terms reactant and reaction product here are each understood to mean the singular and the plural.

In the first reaction unit, as described, the pressure p1 is present. Since the system is closed off from the outside, this pressure essentially exists in the entire reaction unit, aside from fluctuations for process-related reasons. Thus, the reactant in the first reaction unit also has the pressure p1. The pressure reduction apparatus reduces the pressure that acts on the reactant; it is introduced into the second reaction unit at the pressure p2, with the second reaction unit being operated essentially at exactly that pressure p2. Here too, reaction-related local pressure fluctuations may of course occur. A third reaction unit may further be provided, which is operated at a pressure p3, where the pressure p3 is lower again than the pressure p2. This is appropriate because reactant unconsumed even during the reaction in the second reaction unit occurs in the absorption section, which is in turn introduced into the third reaction unit with only a low pressure drop. The difference between the pressures p1, p2 and p3 here is advantageously between 0.5 bar and 10 bar.

In principle, the reaction cascade described may comprise any number of reaction units 1 to n, where the number n of reaction cascades in which the process of the invention is performed is determined by how much unconsumed reactant remains in the reaction section in the respective reaction process, and whether it is economically worthwhile still to transfer this excess reactant to a further reaction unit. The n reaction units are operated here with falling pressure each time from the first to the nth reaction unit.

The reaction section of the reaction unit here advantageously has a tubular configuration, such that the reactant flows through it along its longitudinal extent. The effect of this flow through a tubular reaction section is that excess reactants and products are ultimately guided through the reaction section and, downstream of the reaction section provided with catalyst, can be separated again from one another in the absorption section. Moreover, the flow through the reaction section in longitudinal direction makes it possible to dispense with moving parts in the reaction section, which reduces the manufacturing costs thereof.

Moreover, it is appropriate that the absorbent laden with the reaction product(s) is guided through an absorbent outlet into a desorption unit, where the reaction product is unloaded therefrom. The unladen absorbent can subsequently be introduced back into the absorption section.

Further advantageous configuration forms of the invention and further features will be apparent from the drawings that follow. These are merely schematic drawings that do not constitute any restriction of the scope of protection.

### BRIEF DESCRIPTION OF THE DRAWINGS

The figures show:

FIG. 1: a reaction cascade for implementation of equilibrium-limited reactions and

FIG. 2: a schematic diagram of the processing of the absorbent.

### DETAILED DESCRIPTION OF INVENTION

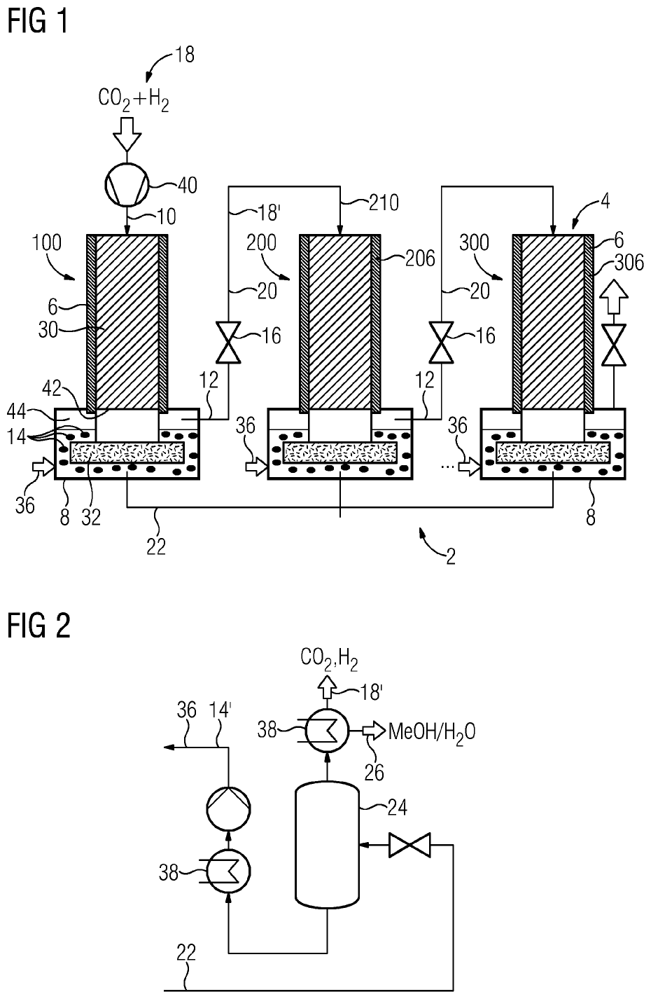

FIG. 1 shows a schematic diagram that serves as an example of a reaction cascade suitable for implementing equilibrium-limited reactions, represented here by the example of carbon dioxide and hydrogen, with minimum loss. In the example described, carbon dioxide and hydrogen as reactant or reactant gas is introduced into a reaction unit 4 at elevated pressure, for example of greater than 30 bar, with the aid of a compressor. More specifically, the reactant 18 is introduced into a reaction section 6 via a reactant inlet 10. A catalytic substance 30 is disposed in the reaction section 6. This catalytic substance 30, also referred to hereinafter as catalyst 30, may be in various configuration forms. In a very appropriate and simple configuration form, the catalyst 30 is in the form of a bed of powder in the reaction section 6. In principle, however, it is also possible to fit porous sintered bodies containing the catalyst 30 at least on the surface into the reaction section 6. It is thus possible to achieve a defined surface area, which, however, also entails higher expenditure in the production of the catalyst 30. The reaction section 6 here is advantageously of tubular configuration, “tubular” being understood to mean that the ratio of length to width, i.e. the aspect ratio of the reaction section 6, is greater than 1, advantageously greater than 5.

At one end of the reaction section 6, the opposite end from the reactant inlet 10, is disposed an absorption section 8, wherein the absorption section 8 and the reaction section 6 are advantageously closely connected with one another in spatial terms. More advantageously, the absorption section 8 is flanged directly onto the reaction section 6 by a flange 42. This construction of the reaction unit 4 can be configured particularly inexpensively. The absorption section 8 here advantageously has a gas filter apparatus 32 that may be configured, for example, in the form of a sintered plate or in the form of a perforated tube. Also present in this absorption section is an absorbent 14. In the diagram according to FIG. 1, the gas filter apparatus 32 is surrounded completely by the liquid absorbent 14.

There follows a description of the reaction process that takes place in the reactor unit in the individual components described, using the example of the carbon dioxide and hydrogen reactant already mentioned. The mixture of carbon dioxide and hydrogen is guided into the reaction section 6, and especially onto the catalytic substance 30 therein. The catalyst 30 has a surface that has catalytic action and converts the carbon dioxide and hydrogen to methanol. However, this reaction has an equilibrium that is established when only 20% of the methanol product has formed. In order to allow the reactions to continue, it is necessary for the product to be constantly removed from the reaction site, i.e. the surface of the catalyst 30, and new reactant to be supplied. This is achieved by the flow of the reactant through the tubular reactor section 6, in the course of which the resultant methanol product, which is liquid under the process conditions of about 30 to 50 bar and a temperature of more than 200 degrees Celsius, is formed in each case. Thus, the stream of the reactant 18 also entrains the reaction product 26, and introduces it continuously from the reaction section 6 into the absorption section 8. The reaction product 26 and the excess reactant 18 are then present together in gaseous form therein, in the form of the gas mixture of carbon dioxide and hydrogen. This gaseous mixture of reactant 18 and product 26 is guided through the gas filter apparatus 32, with absorption of the product 26, the methanol in the example specified, by the absorbent 14, generally or advantageously in the form of an ionic liquid. The gaseous reactants 18 are selectively not absorbed by the absorbent 14 and collect in a gas space 44 of the absorption section 8. From the gas space 44 of the absorption section 8, a connecting conduit 20 is provided, in which or on which a pressure reduction valve 16 is provided. The excess reactant 18, which is in the form of a pressure p1 in the reaction section, is reduced by the pressure reduction valve 16 to a pressure p2, and is introduced into a second reaction unit 200 in the form of a reactant 18′.

In the second reaction unit 200, by contrast with the first reaction unit 100, there is a reaction pressure p2 which is about 2 bar lower than the reaction pressure p1 at which the first reaction unit 100 is operated. The reduction in the process pressure p by about 2 bar, for example from 50 bar to 48 bar, leads merely to a comparatively small loss of efficiency in the performance of the equilibrium-limited reaction, as already described with regard to the first reaction unit 100. However, the effect of the pressure reduction is that it is not necessary to recompress the recovered or excess reactant 18 by an energy-intensive and technically costly compression operation. The pressure employed in the next reaction unit is merely that at which the reactant already exists in any case, and the reaction described is conducted again with slightly altered thermodynamic parameters. The result is a reaction cascade 2 having at least two reaction units 4, 100, 200, where the ultimate number n of reaction units 4 is determined by process-related boundary conditions and is set according to the conversion, total volume of the reaction units and product demand, and also according to economic considerations. It should be stated here that the configuration of the reaction unit 4 or 100 and 200 is technically relatively favorable since it is possible to dispense with moving parts, for example stirrers that have to be driven and have bearing devices. In the present configuration form according to FIG. 1, it is possible to dispense with moving parts apart from the first compressor 40 that compresses the reactant into the first reaction unit 100. The reaction cascade 2 shown in FIG. 1 has three reaction sections 4 in this case, this being a purely illustrative schematic representation. Moreover, the reaction units 4, 100, 200 and 300 are shown in equal size. They are also shown as being of the same type. This has the advantage that mass production of multiple reaction units 4 can likewise again be configured in an inexpensive manner. In principle, the reaction units 100, 200, 300 along the cascade 2 can be reduced in terms of their reaction volume. In this case, however, it is merely the volume of the reaction unit or of the reaction section and possibly also of the absorption section 8 that is reduced, but there is little change in the design thereof. The reason for the reduction in the reaction volume is that the reactant 18 is introduced only once into the cascade in the configuration envisaged. Thus, no further reactant is introduced during the progress of the reaction in the downstream reaction units 200 and 300, since this would mean further energy expenditure by compression of the base reactant 18. Thus, even within the cascade 2, the volume of the reactant 18 available decreases in the further reaction units 200 and 300, and therefore the reaction volume in the reaction section 206 and 306 of the reaction units 200 and 300 can also be reduced gradually.

FIG. 2 illustrates the circulation of the absorbent 14, specifically in the phase in which it leaves the absorption section 8 at the absorbent outlet 22. A desorption unit 24 is provided, in which the absorbent 14 laden with the reaction product 26 is freed therefrom. This “regeneration” of the absorbent 14 can be effected by lowering the pressure and/or increasing the temperature. The introduction of what is called a stripping gas for desorption may also be appropriate. The gas that has thus been freed of the absorbent 14 and contains the reaction products 26 is subsequently guided into a heat exchanger 38 in which the reaction product 26, for example methanol, is separated by condensation from the remaining gaseous constituents, especially comprising the reactant gases carbon dioxide and hydrogen. The reaction products, especially the methanol which, however, also contains water, can be removed for further processing. The reactants 18 or 18′ that have likewise been recovered therefrom can be fed back to the process, and introduced into the first reaction unit 100 via the compressor 40. The unladen absorbent, labeled 14′ here, is heated and introduced as unladen absorbent 14 via an absorbent feed 36 back into the absorption section 8.

The reaction of carbon dioxide and hydrogen to give methanol and water that proceeds over the catalytic substance 30 in the reaction section 6 is exothermic. This means that the reaction section 6 heats up. Countercurrent cooling through an outer wall of the reaction section 6 is appropriate here. The reaction section 6 here is advantageously of jacketed design in terms of its outer shell. The thermal energy obtained thereby can be used for heating in some other way, for example for heating of the reactant gas 18. It is also possible to use the energy possessed by the absorbent 14 after unloading for this purpose.

### LIST OF REFERENCE NUMERALS

2 reactor cascade
4 reactor unit
6 reaction section
8 absorption section
10 reactant inlet
12 reactant outlet
14 absorbent
16 pressure reduction valve
18 reactants
20 connecting conduit
100 first reactor unit
200 second reactor unit
210 reactant inlet to reactor unit
22 absorbent outlet
24 desorption unit
26 reaction products
28 reaction volume
106 reaction section of first reaction unit
206 reaction section of second reaction unit
30 catalytic substance
32 gas filter apparatus
34 longitudinal extent of reactor section
36 absorbent feed
38 heat exchanger
40 heat exchanger
42 flange
44 gas space of identical reactor

1. A reactor cascade for implementing equilibrium-limited reactions, comprising:
at least two reactor units each comprising a reaction section in the form of a tubular reactor and each comprising an absorption section,
wherein the reaction section has a reactant inlet and the absorption section has a reactant outlet for leading off excess reactants,
a connecting conduit between the reactant outlet of a first reactor unit and the reactant inlet of a second reactor unit, and
a pressure reduction valve for reduction of a process pressure p between the first reactor unit and the second reactor unit.

2. The reactor cascade as claimed in claim 1,
wherein the reactant inlet is provided at one end of the reaction section, and the absorption section is disposed at another end.

3. The reactor cascade as claimed in claim 1,
wherein the absorption section has an absorbent outlet as well as the reactant outlet.

4. The reactor cascade as claimed in claim 3,
wherein the absorbent outlet is connected to a desorption unit for unloading reaction products from an absorbent.

5. The reactor cascade as claimed in claim 1,
wherein a reaction section of the first reactor unit has a higher reaction volume than a reaction section of a second reactor unit.

6. The reactor cascade as claimed in claim 1,
wherein the reactor units are of identical design.

7. The reactor cascade as claimed in claim 1, further comprising:
a gas filter apparatus disposed in the absorption section.

8. A process for performing an equilibrium-limited reaction, the process comprising:
guiding a reactant into a reaction section of a reactor unit at least partly filled with a porous catalytic substance through which the reactant flows, wherein the reactant is at least partly converted to a reaction product at a surface of the porous catalytic substance,
guiding the reaction product and excess reactant from the reaction section into an absorption section of the reactor unit, wherein the reaction product is absorbed by the absorbent and the excess reactant is separated from the reaction product by means of a gas filter apparatus, and wherein there is a pressure p1 in the reactor unit, and
guiding the separated reactant through a pressure reduction apparatus and introducing the separated reactant into a second reactor unit at a pressure p2, where the pressure p2 is less than the pressure p1.

9. The process as claimed in claim 8,
wherein the second reactor unit is operated at the pressure p2.

10. The process as claimed in claim 8, further comprising:
a third reactor units operated at a pressure p3 lower than the pressure p2.

11. The process as claimed in claim 8,
wherein a reactor cascade of at least two reactor units is provided, which are operated with a falling operating pressure pn proceeding from a first reactor unit.

12. The process as claimed in claim 11,
wherein the reaction section has a tubular configuration and the reactant flows through the reaction section along its longitudinal extent.

13. The process as claimed in claim 8,
wherein the reaction product comprises methanol.

14. The process as claimed in claim 8,
wherein the reactant comprises carbon dioxide and hydrogen.

15. The process as claimed in claim 8,
wherein the absorbent laden with the reaction product is guided through an absorbent outlet into a desorption unit, where the reaction product is unloaded therefrom.
